(19) United States
(12) Patent Application Publication
(10) Pub. No.: US 2024/0389903 A1
(43) Pub. Date: Nov. 28, 2024

(54) ASSEMBLY HAVING A DETECTOR

(71) Applicant: Heidelberg Engineering Gmbh, Heidelberg (DE)

(72) Inventors: Christoph BROSCHE, Eppelheim (DE); Tobias HARION-NIKITKA, Heidelberg (DE)

(73) Assignee: Heidelberg Engineering Gmbh, Heidelberg (DE)

(21) Appl. No.: 18/699,335

(22) PCT Filed: Aug. 19, 2022
(86) PCT No.: PCT/EP2022/073182

§ 371 Date: Apr. 8, 2024

(30) Foreign Application Priority Data
Oct. 8, 2021 (DE) 10 2021 126 145.7

(51) Int. Cl. A61B 5/1455; A61B 3/12; G01N 21/64

(52) U.S. Cl. CPC A61B 5/14555; A61B 3/12; A61B 5/14552; G01N 21/6486

(57) ABSTRACT

An assembly, in particular for use in a confocal laser scanning apparatus, comprising a light-sensitive detector (1) and a device (2) for focusing and/or geometrically constraining light onto a detection path (3), is disclosed in response to the problem of specifying an assembly having a detector, which assembly can generate images as quickly as possible at high light sensitivity, characterised in that a device (4) is provided in the detection path (3) and spectrally splits the light, wherein at least one optical component (5) is provided, which directs the spectrally split light onto a detection row (6) of the detector (1), the detection row (6) having fields (6i) onto each of which light of different wavelengths is incident.

The invention relates to an assembly according to the preamble of claim 1.

Assemblies are already known, which comprise a light-sensitive detector and an apparatus for focusing and/or geometrically delimiting light on a detection path. In particular in conventional laser scanning systems, various technologies can be used for spectrally sensitive light detection.

Thus, for example, using a single detector for non-simultaneous imaging is known, wherein the colors red, green, and blue are acquired alternately. Using a filter wheel for a single detector having a blocking filter for fluorescence imaging is also known. Furthermore, distributing light via a beam splitter onto multiple detection channels of a detector is known. Using a spectrometer having a grating for spectral distribution of light onto very many fields of a detector of equal size is also known.

Some known technologies are subject to the disadvantage that a relatively slow recording speed is provided in the imaging. Furthermore, it can be disadvantageous in some technologies that light is partially blocked and cannot be used for imaging. Some technologies are also optically relatively complex to implement and are limited with regard to the number of detection channels to be used in the imaging.

The invention is therefore based on the object of specifying an assembly having a detector, which can generate images as quickly as possible with high light sensitivity.

The present invention achieves the above-mentioned object by way of the features of claim 1.

It has first been recognized that some known technologies are complex, costly, and/or generally have relatively little light sensitivity and therefore there is a need for a technology which implements a high speed in the imaging by parallel use of detector channels. Furthermore, it has been recognized that light losses due to blocking filters are to be avoided as much as possible or are to be completely avoided. Furthermore, it has been recognized that there is a need for relatively simple and compact designs, even if they have many detector channels for imaging. It has also been recognized that there is a need for a relatively simply designed spectrometer in which a lower optical and spectral resolution is required to achieve a high light sensitivity relatively without problems.

Against this background, it has been recognized that with a technology which avoids the disadvantages of the prior art, an apparatus has to be provided in a detection path, by means of which light can be spectrally split, wherein at least one optical component is provided, by means of which the spectrally split light can be guided onto a detection line of a detector. Light can be split into light beams having different wavelengths by the spectral splitting. A broad color spectrum can thus be generated, the individual wavelengths of which can be assigned to different areas on the detection line. It has been recognized therefrom that the detection line has to have fields, on each of which light having different wavelengths is incident, in order to thus be able to analyze and evaluate individual fields separately from one another. A large number of wavelengths of the light can thus be detected simultaneously with high sensitivity and processed for imaging.

Due to the mentioned assembly, a mechanical filter wheel is advantageously not required, and light is also not lost due to blocking filters. A simultaneous recording of various image modes without speed loss is possible, and images of various image modes can be recorded exactly simultaneously, so that they are combinable and comparable to one another better than using assemblies of the prior art.

The mentioned assembly is very flexible, because the mentioned fields are assignable to spectral segments, the geometries or light sensitivities of which can be adapted to the requirements of various applications.

Such spectral segments from fields of the detection line can be read out individually or completely interconnected or partially interconnected.

In comparison to a standard spectrometer, a device having an assembly of the type described here can manage with substantially fewer segments or spectral ranges, which are relevant for the application, however.

In addition, a significantly lower spectral resolution is necessary, for example, only six spectral segments can be provided. A lower optical resolution is advantageously required, and the assembly is more light-sensitive than a conventional line camera.

Against this background, the detector could be designed as a line detector, the detection line of which can be dynamically divided into individual spectral segments by an analog or digital circuit, wherein each spectral segment can be configured for acquiring or with regard to the sensitivity of a specific spectral range and/or can be configured with regard to its spectral quantum efficiency. In this way, the mentioned assembly is very flexible due to the use of suitable electronics, because each segment can be adapted to the requirement of a specific application solely electronically. The assembly does not have to be adapted by changing the optical structure, in particular of a beam path, physically to specific applications.

The fields of the detection line could be assigned to multiple successive spectral segments or form such spectral segments. The spatial or geometric extension of a spectral segment can thus be set. Alternatively or additionally, the detection line could be divided into different spectral segments which each acquire light from different spectral ranges or having different wavelengths. The detection line can thus be adapted to the expected light to be acquired or its wavelength distribution. Individual spectral segments can be created which are sensitive to very specific light wavelength ranges or spectral ranges.

At least two or more spectral segments could differ from one another in their size or spatial, in particular linear and/or planar, extension, so that spectral segments of different sizes detect light from spectral ranges extending by different amounts or from intervals of the wavelengths of different sizes.

Moreover, it is conceivable to adapt the detection line to an intensity distribution of light having different wavelengths. Spectral ranges having expected lower intensity could be acquired, for example, by larger spectral segments in order to thus increase the light sensitivity. The detection line can be adapted to the spectral splitting behavior of the apparatus for spectrally splitting light, for example, to the characteristics of a prism.

A readout apparatus could be provided, which interconnects individual fields of the detection line in an analog or digital manner to form a spectral segment or to form multiple spectral segments. Alternatively or additionally, the readout apparatus could read out the detection signals of a spectral segment individually or read out the detection signals of all spectral segments completely interconnected or read out the detection signals of individual spectral segments interconnected to form a group.

Against this background, the readout apparatus could process the detection signals and/or interconnect them to form an output channel and process them together using an image creation apparatus, which creates one image or multiple images which is or are generated on the basis of detection signals obtained in parallel and/or simultaneously from one spectral segment or multiple spectral segments. Rapid imaging with high specific light sensitivity is thus implemented.

The apparatus for delimiting light on the detection path could be designed as a perforated screen. A point detection in a confocal system is possible in this way. The detector is usable as a point detector. A perforated screen is also designated as a “pinhole” in English.

The apparatus for spectrally splitting the light could be designed as a prism or a grating. A prism is mechanically robust and can be turned, rotated, or inclined without problems within an assembly. A grating can be used in a particularly space-saving manner in an assembly.

The optical component could be designed as a lens or as a successive connection of multiple lenses, preferably multiple cylinder lenses. Focusing of light is possible without problems by a lens.

The detection line could be manufactured, for example, from silicon photomultipliers (SiPM). These apparatuses have proven to be particularly reliable in order to reliably detect light with high sensitivity.

A device for simultaneously recording and/or displaying one image or multiple images on the basis of simultaneously detected light of various spectral ranges could have an assembly of the type described here.

The device could simultaneously record and/or display true color images from three color channels, preferably from the color channels red, green, and blue (R, G, B). Rapid color imaging is thus enabled. In the same manner, the device is also capable of acquiring images outside the visible wavelength range (for example infrared).

The device could simultaneously record and/or display fluorescence and reflection images. Various light sources can be used in the optical acquisition of organic structures in this way. Images for diagnostic purposes and images for orientation purposes can be recorded and displayed simultaneously during the examination of organic tissue.

The assembly described here or the above-mentioned device could be used to carry out oximetry, namely to detect oxygen in human or animal blood.

The assembly described here or the above-mentioned device could be used to carry out a spectrally resolved autofluorescence analysis in order to identify substances and/or to acquire structures, in particular in human or animal tissue.

The assembly described here or the above-mentioned device could be used in a fluorescence lifetime imaging ophthalmoscope (FLIO).

The detector described here is a photo detector, which can advantageously operate in a spectrally sensitive manner like a line camera having variable segment size. The detector advantageously comprises a photodetector array for spectrally sensitive light detection.

In the figures of the drawing

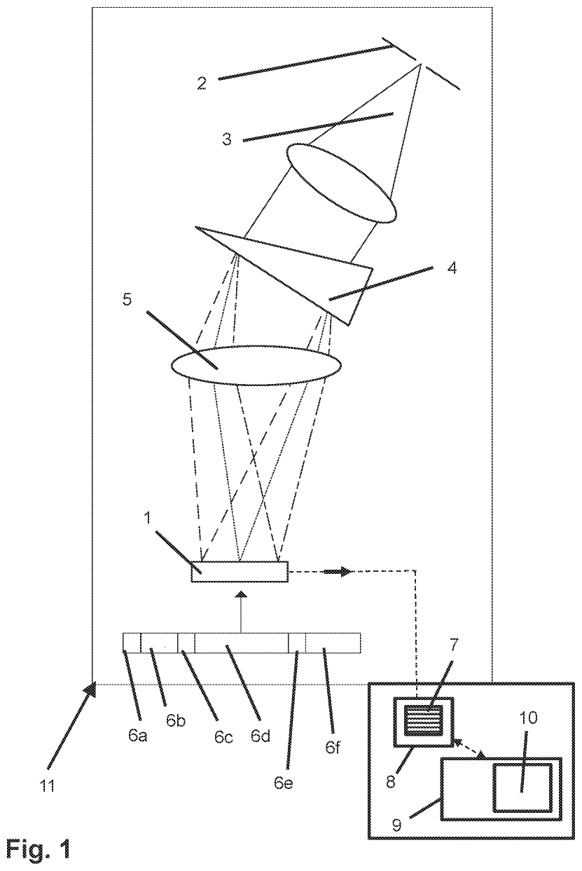

FIG. 1 shows a schematic representation of a device which has an assembly which comprises a detector having a detection line that has fields on each of which light having different wavelengths is incident, and

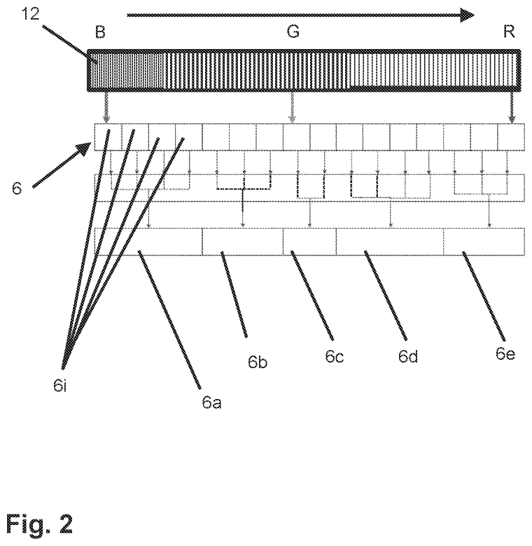

FIG. 2 shows a schematic representation of a detection line in which different fields are combined to form different spectral segments of different sizes, and the top of FIG. 2 shows a schematic representation of a light spectrum which extends continuously from the color blue via the color green to the color red in color graduations, after the light has been split by a prism according to FIG. 1.

FIG. 1 insofar shows an assembly, in particular for use in a confocal laser scanning device, comprising a light-sensitive detector 1 and an apparatus 2 for focusing and/or geometrically limiting light to a detection path 3. An apparatus 4 is provided in the detection path 3, which spectrally splits the light, wherein at least one optical component 5 is provided which guides the spectrally split light onto a detection line 6 of the detector 1. FIG. 2 shows, as a supplement to FIG. 1, that such a detection line 6 has fields 6i, on each of which light having different wavelengths is incident.

FIG. 1 shows that the detector 1 is designed as a line detector, the detection line 6 can be dynamically divided by an analog or digital circuit 7 into individual spectral segments 6a-f, wherein each spectral segment 6a-f can be configured to acquire or with regard to the sensitivity of a specific spectral range and/or can be configured with regard to its spectral quantum efficiency.

The fields 6i of the detection line 6 according to FIG. 1 or FIG. 2 are electronically assigned to multiple successive spectral segments 6a-f or 6a-e and form such spectral segments 6a-f or 6a-e. According to FIG. 1, only six spectral segments 6a-f are provided. FIG. 2 shows a detection line 6 having only five spectral segments 6a-e.

The detection line 6 according to FIG. 1 or FIG. 2 is divided into various spectral segments 6a-f or 6a-e, which each acquire light from different spectral ranges or having different wavelengths. All fields 6i are equal in size.

FIGS. 1 and 2 show that at least more than two spectral segments 6a-f or 6a-e differ from one another in their size or spatial, namely linear and/or planar, extension, so that spectral segments 6a-f or 6a-e of different sizes detect light from spectral ranges extending by different amounts or from intervals of different sizes of the wavelengths of the split light.

The spectrum 12 having blue, green, and red spectral ranges is schematically shown at the top in FIG. 2. It is shown farther down in FIG. 2 that the first spectral segment 6a comprises four fields 6i and the spectral segment 6b adjoining thereon only comprises three fields 6i.

The first spectral segment 6a having four fields 6i detects light having a wavelength interval from the blue spectral range, the fifth spectral segment 6e having three fields 6i detects light having a wavelength interval from the red spectral range.

FIG. 1 also shows that a readout apparatus 8 is provided, which electronically interconnects individual fields 6i of the detection line 6 in an analog or digital manner to form a spectral segment 6a-f or to form multiple spectral segments 6a-f.

The readout apparatus 8 can read out the detection signals of a spectral segment 6a-f individually or can read out the detection signals of all spectral segments 6a-f completely interconnected or can read out the detection signals of individual spectral segments 6a-f interconnected to form a group.

With application of one and/or more of the above-mentioned possibilities, the readout apparatus 8 processes the detection signals and/or interconnects them to form an output channel and cooperates with an image creation apparatus 9, which creates one image 10 or multiple images 10, which is or are generated on the basis of detection signals obtained in parallel and/or simultaneously from one spectral segment 6a-f or multiple spectral segments 6a-f.

Specifically, the apparatus 2 for limiting light to the detection path 3 is designed as a perforated screen. The apparatus 4 for spectrally splitting the light is designed as a prism. The light is spectrally decomposed in the assembly functioning as the detection unit after the perforated screen and imaged via an optical component 5 on the detection line 6.

The detection line 6 according to FIG. 1 consists of multiple, in the specific example six, light-sensitive spectral segments 6a-f which are concatenated directly, nearly continuously. Individual spectral segments 6a-f differ with respect to their size from one another and accordingly cover spectral ranges of different sizes. The spectral segments 6a-f can be read out individually, completely interconnected, or partially interconnected.

The optical component 5 is designed as a lens. The detection line 6 is manufactured from silicon photomultipliers (SiPM).

FIG. 1 shows a device 11 for simultaneously recording and/or displaying one image 10 or multiple images 10 on the basis of simultaneously detected light of various spectral ranges, which has an assembly of the above-described type.

The device 11 simultaneously records true color images from three color channels, preferably from the color channels red, green, and blue (R, G, B) and/or displays them. Simultaneously, further nonvisible wavelengths (such as infrared and/or ultraviolet) can also be acquired.

The device 11 can simultaneously record and/or display fluorescence and reflection images. The dynamic configuration of the spectral segments 6a-f or 6a-e in order to adapt them to the respective application or the relevant spectral range is of particular importance here.

The compilation of the detector 1 of the device in spectral segments 6a-f or 6a-e is dynamically implemented by an analog or digital circuit. A detector having fixed or unchangeable spectral segments is thus not required for different applications, but rather the detector 1 may be dynamically configured for any application.

### LIST OF REFERENCE SIGNS

1 detector
2 apparatus for focusing
3 detection path
4 apparatus for spectral splitting
5 optical component
6 detection line
6i field of 6
6a-f spectral segment
7 circuit
8 readout apparatus
9 image creation apparatus
10 image
11 device
12 spectrum from blue to red

1. An assembly, in particular for use in a confocal laser scanning device, comprising a light-sensitive detector and an apparatus for focusing and/or geometrically delimiting light on a detection path,
wherein
an apparatus is provided in the detection path, which spectrally splits the light, wherein at least one optical component, which guides the spectrally split light onto a detection line of the detector, is provided, and wherein the detection line has fields, on each of which light having different wavelengths is incident.

2. The assembly as claimed in claim 1, wherein the detector is designed as a line detector, the detection line of which can be dynamically divided into individual spectral segments by an analog or digital circuit, wherein each spectral segment can be configured to acquire or with regard to the sensitivity of a specific spectral range and/or can be configured with regard to its spectral quantum efficiency.

3. The assembly as claimed in claim 1, wherein the fields of the detection line are assigned to multiple successive spectral segments or form such spectral segments and/or wherein the detection line is divided into different spectral segments, which each acquire light from different spectral ranges or having different wavelengths.

4. The assembly as claimed in claim 2, wherein at least two or more spectral segments differ from one another in their size or spatial, in particular linear and/or planar, extension, so that spectral segments of different sizes detect light from spectral ranges extending by different amounts or from intervals of the wavelengths of different sizes.

5. The assembly as claimed in claim 1, wherein a readout apparatus is provided, which interconnects individual fields of the detection line in an analog or digital manner to form a spectral segment or to form multiple spectral segments, and/or in that the readout apparatus reads out the detection signals of a spectral segment individually or reads out the detection signals of all spectral segments completely interconnected or reads out the detection signals of individual spectral segments interconnected to form a group.

6. The assembly as claimed in claim 5, wherein the readout apparatus processes the detection signals and/or interconnects them to form an output channel and cooperates with an image creation apparatus, which creates one image or multiple images, which is or are generated on the basis of detection signals obtained in parallel and/or simultaneously from one spectral segment or multiple spectral segments.

7. The assembly as claimed in claim 1, wherein the apparatus for delimiting light on the detection path is designed as a perforated screen.

8. The assembly as claimed in claim 1, wherein the apparatus for spectrally splitting the light is designed as a prism or a grating.

9. The assembly as claimed in claim 1, wherein the optical component is designed as a lens or a connection in succession of multiple lenses, preferably multiple cylinder lenses.

10. The assembly as claimed in claim 1, wherein the detection line is manufactured from silicon photomultipliers (SiPM).

11. A device for simultaneously recording and/or displaying one image or multiple images on the basis of simultaneously detected light of various spectral ranges, which has an assembly as claimed in claim 1.

12. The device as claimed in claim 11, wherein the device simultaneously records and/or displays true color images from three color channels, preferably from the color channels red, green, and blue.

13. The device as claimed in claim 11, wherein the device simultaneously records and/or displays fluorescence and reflection images.

14-16. (canceled)

17. A method for carrying out oximetry to acquire oxygen in human or animal blood, the method comprising: providing an assembly comprising a light-sensitive detector (1) and an apparatus (2) for focusing and/or geometrically delimiting light on a detection path (3), wherein an apparatus (4) is provided in the detection path (3), which spectrally splits the light, wherein at least one optical component (5), which guides the spectrally split light onto a detection line (6) of the detector (1), is provided, and wherein the detection line (6) has fields (6i), on each of which light having different wavelengths is incident; and using the assembly to acquire oxygen in human or animal blood.

18. A method for carrying out a spectrally resolved autofluorescence analysis to identify substances and/or to acquire structures in human or animal tissue, the method comprising: providing an assembly comprising a light-sensitive detector (1) and an apparatus (2) for focusing and/or geometrically delimiting light on a detection path (3), wherein an apparatus (4) is provided in the detection path (3), which spectrally splits the light, wherein at least one optical component (5), which guides the spectrally split light onto a detection line (6) of the detector (1), is provided, and wherein the detection line (6) has fields (6i), on each of which light having different wavelengths is incident; and using the assembly to identify substances and/or to acquire structures in human or animal tissue by carrying out a spectrally resolved autofluorescence analysis.

19. A method for using an assembly in a fluorescence lifetime imaging ophthalmoscope (FLIO), the method comprising: providing the assembly comprising a light-sensitive detector (1) and an apparatus (2) for focusing and/or geometrically delimiting light on a detection path (3), wherein an apparatus (4) is provided in the detection path (3), which spectrally splits the light, wherein at least one optical component (5), which guides the spectrally split light onto a detection line (6) of the detector (1), is provided, and wherein the detection line (6) has fields (6i), on each of which light having different wavelengths is incident; and using the assembly in the fluorescence lifetime imaging ophthalmoscope (FLIO).
